(19) United States
(12) Patent Application Publication
(10) Pub. No.: US 2012/0055484 A1
(43) Pub. Date: Mar. 8, 2012

(54) Ventilator

(76) Inventors: Geoffrey Mark Shaw, Christchurch (NZ); Geoffrey Anders, Christchurch (NZ); Lawrence Gordon Alloo, Dunedin (NZ)

(21) Appl. No.: 13/319,690

(22) PCT Filed: May 14, 2010
(86) PCT No.: PCT/IB2010/052140

§ 371 Date: Nov. 10, 2011

(30) Foreign Application Priority Data
May 15, 2009 (NZ) 577026

(51) Int. Cl. A61M 16/00; A61M 16/04; F04B 49/20; A61M 16/06

(52) U.S. Cl. 128/206.21; 128/200.24; 128/207.14; 417/42

(57) ABSTRACT

A ventilator which includes: at least one pair of reciprocating opposed gas moving means, the internal volume of which is arranged to be reduced and enlarged by a balanced drive, said gas moving means being arranged to move gas at a predetermined pressure and/or volume to a delivery tube; control means for regulating the speed and the distance of movement of the drive; wherein the masses of all moving parts are balanced so as to minimise the vibration of the ventilator in use.

### TECHNICAL FIELD

The present invention relates to a ventilator which is capable of providing, continuous positive airway pressure (CPAP), assisted spontaneous breathing (ASB), all forms of mandatory ventilation (MV), and high frequency ventilation (HFV). All of these ventilation modes are known.

CPAP typically supplies gas at a pressure in the range 2.5 cm water to 15 cm water, at a frequency controlled by the patient and typically in the range 8 to 30 breaths per minute.

ASB typically supplies gas at a pressure in the range 5 cm water to 15 cm water above the expired pressure (which is typically 2.5 cm water to 15 cm water), with the frequency controlled by the patient as for CPAP; typical volumes for ASB are in the range 6 to 10 mL per kilogram of predicted patient weight. ASB may include, but is not limited to, the following modes of ventilation: bilevel, airway pressure release, proportional assist, pressure support, or volume assured ventilation.

Mandatory ventilation (MV) typically supplies gas at a pressure of up to 35 cm water, with a positive expiratory pressure of up to 20 cm water; typically, the frequency is in the range 8 to 30 breaths per minute. MV may include, but is not limited to, synchronised intermittent ventilation, which may be either pressure or volume controlled, or adaptive supportive ventilation, in which the tidal volume and frequency are varied according to the patient's condition, to minimise the work of breathing.

HFV typically supplies gas at a pressure in the range 10 to 30 cm water, and the pressure difference between inspiration and expiration typically is about 60 cm water. Frequencies typically are in the range 3 to 6 Hz for an adult and up to 15 Hz for a neonate. The term HFV is used for both high-frequency jet ventilation and high-frequency oscillatory ventilation, but the apparatus of the present invention is limited to the provision of high frequency oscillatory ventilation (HFOV), in which there is active controlled inspiration and expiration.

As used herein, the term “ventilator” means any type of equipment designed to supply pressurised gas for breathing to a patient. Pressurised gas may be simply pressurised air or may be pressurised air enriched with additional oxygen and/or other gases, or may be in mixture of gases other than air e.g. helium/oxygen mixtures. The patient may be capable or incapable of breathing independently.

### BACKGROUND ART

Any discussion of the prior art throughout the specification is not an admission that such prior art is widely known or forms part of the common general knowledge in the field.

A range of different types of equipment currently is available for providing ventilation, but in general the equipment is specifically designed either to provide CPAP, ASB, MV or HFV; no ventilator currently available on the market is capable of delivering all modes of ventilation without compromising one or more of the modes.

When a patient is breathing spontaneously, but requires mechanical ventilation assistance, support is given in the form of CPAP, or ASB (either solely or in combination with a mandatory ventilation mode). When a patient takes a spontaneous breath the ventilator must synchronise with the patient's breathing cycle and it follows that the ventilator must be able to sense demand and delivery accurately, because any failure to synchronise accurately with the patient will both increase the breathing work for the patient and cause discomfort.

When a patient is not capable of breathing spontaneously, the ventilator must be capable of breathing for the patient, i.e. pushing the air or oxygen-enriched air mixture into the patient's lungs; the gases are then exhausted by the lungs. For this, mandatory ventilation or high-frequency ventilation or mandatory ventilation with superimposed high-frequency ventilation, may be used.

### DISCLOSURE OF INVENTION

An object of the present invention is the provision of a ventilator which is capable of providing, with equal efficiency, a range of different modes of ventilation, (e.g. CPAP, ASB, MV, HFOV, or any combination of these modes) and HFOV superimposed upon mandatory (conventional) ventilation, without requiring major reconfiguration of the ventilator when switching between different modes. A further object of the present invention is the provision of a ventilator capable of being used in combination with a conventional ventilator, to superimpose HFOV on conventional ventilation.

The present invention provides a ventilator which includes: at least one pair of reciprocating opposed gas moving means, the internal volume of which is arranged to be reduced and enlarged by a balanced drive, said gas moving means being arranged to move gas at a predetermined pressure and/or volume to a delivery tube; control means for regulating the speed and the distance of movement of the drive; wherein the masses of all moving parts are balanced so as to minimise the vibration of the ventilator in use.

The gas moving means may be any suitable receptacle, for example bellows or cylinders and pistons. Any number of pairs of gas moving means may be used, but the preferred arrangement is two spaced pairs of gas moving means.

The balanced drive may be any suitable linear or rotary balanced drive which is capable of a fast response to the control means. Preferably, the balanced drive consists of a linear motor (linear induction motor) of known type. Another suitable linear drive is a hydraulic or pneumatic ram. A suitable rotary balanced drive would be a pair of matched stepper motors.

Preferably, the balanced drive is a linear balanced drive which is arranged in two parts: a first part which is connected by first connection means to one gas moving means of the or each pair of gas moving means, and a second part which is connected by second connection means to the other gas moving means of the or each pair of gas moving means; said the first and second parts being such that linear movement of either part of said balanced drive producers an equal but opposite movement of the other part of said balanced drive.

Preferably, said first and second parts are linked by a centering device such as a lazy tongs linkage.

Preferably, said balanced drive is mounted upon rails and is arranged to move upon said rails when reciprocating.

Preferably, said at least one pair of opposed gas moving means comprises two pairs of opposed gas moving means, arranged with the longitudinal axis of the gas moving means of each pair aligned with each other, said pairs being spaced apart and having said balanced drive mounted between said pairs.

Preferably, the mass of said first part of said balanced drive and said first connection means is substantially equal to the mass of said second part of said balanced drive and said second connection means, so as to minimise vibration of the ventilator in operation.

In one embodiment of the invention, said gas moving means comprise cylinders and pistons and said at least one pair of opposed gas moving means moving means comprises two pairs of opposed pistons and cylinders arranged with the longitudinal axes of the cylinders of each pair aligned with each other, said pairs being spaced apart and having a balanced drive in the form of a linear induction motor mounted equidistantly between said pairs; said linear induction motor including a stator and a slider coaxial with the stator; wherein said stator and said slider are arranged such to be reciprocated relative to each other and such that linear movement of said stator or said slider produces an equal but opposite movement of said slider or said stator; said stator being connected to one piston of each pair of pistons by first connecting means arranged such that reciprocation of said stator reciprocates each said piston within the corresponding cylinder; and said slider being connected to the other piston of each pair of pistons by second connecting means arranged such that reciprocation of said slider reciprocates each said piston within the corresponding cylinder.

The present invention further provides a ventilation delivery system which includes a ventilator in accordance with the present invention, a gas supply, a gas delivery hose (preferably wide bore) and patient gas delivery means.

### BRIEF DESCRIPTION OF THE DRAWINGS

By way of example only, preferred embodiments of the present invention are described in detail with reference to the accompanying drawings in which:

FIG. 1 is an isometric view of a ventilator in accordance with a first embodiment of the present invention, with the supporting tray omitted for clarity;

FIG. 2 is a plan view of the ventilator of FIG. 1;

FIG. 3 is an end view of the ventilator of FIG. 2 viewed in the direction of arrow A;

FIG. 4 is an end view of the ventilator of FIG. 2 viewed in the direction of arrow B;

FIG. 5 is a flow chart showing part of a patient delivery system;

FIG. 6 is an isometric view of a ventilator in accordance with a second embodiment of the present invention, with the supporting tray omitted for clarity;

FIG. 7 is a plan view of the ventilator of FIG. 6, but at a different stage in the cycle of operation;

FIG. 8 is an end view of the ventilator of FIG. 6 viewed in the direction of arrow G.;

FIG. 9 is an end view of the ventilator of FIG. 6 viewed in the direction of arrow H;

FIG. 10 is an isometric view of a ventilator in accordance with a third embodiment of the present invention, with the supporting tray omitted for clarity;

FIG. 11 is a plan view of the ventilator of FIG. 10;

FIG. 12 is an end view of the ventilator of FIG. 10 viewed in the direction of arrow J;

FIG. 13 is an end view of the ventilator of FIG. 10 viewed in the direction of arrow K; and

FIG. 14 is a diagrammatic plan view of a further type of balanced drive.

### BEST MODE FOR CARRYING OUT THE INVENTION

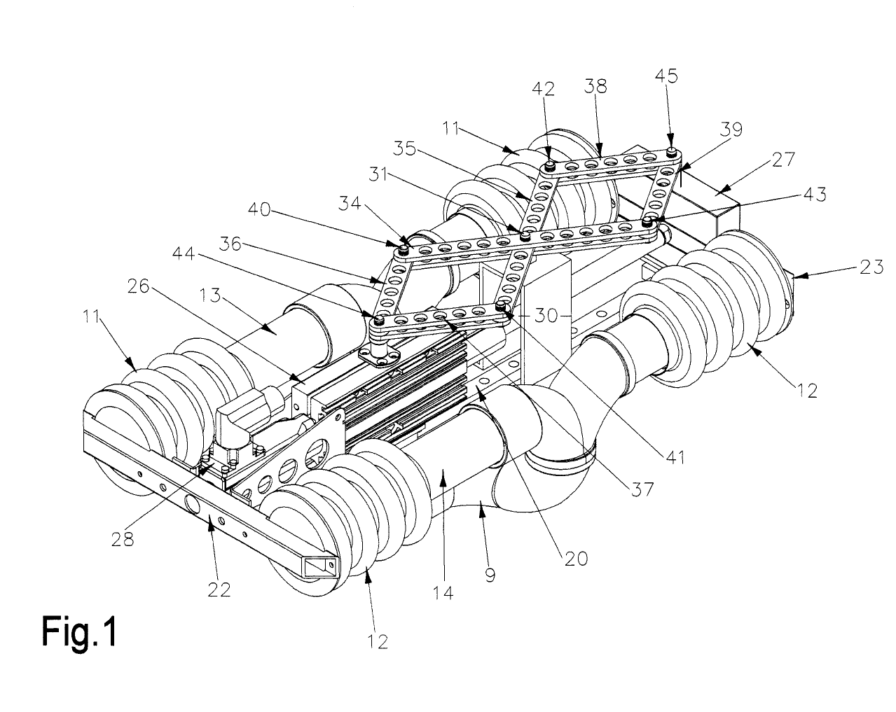

Referring to FIGS. 1 to 4 of the drawings, a ventilator 10 in accordance with a first embodiment of the present invention includes two pairs of opposed gas moving means in the form of bellows 11,12, each bellow of each pair of bellows being connected at one end to an outlet tube 13,14 which join into a common outlet 9. The bellows pairs 11,12 are spaced apart and a linear motor 15 is mounted between the pairs of bellows. As used herein, the term “linear motor” means a linear induction motor. Any suitable linear motor in the known range of linear induction motors may be used; this may include a pair of linear motors, each acting on one opposing set of bellows such that there is minimal net movement of the apparatus. Linear motors are electronically powered.

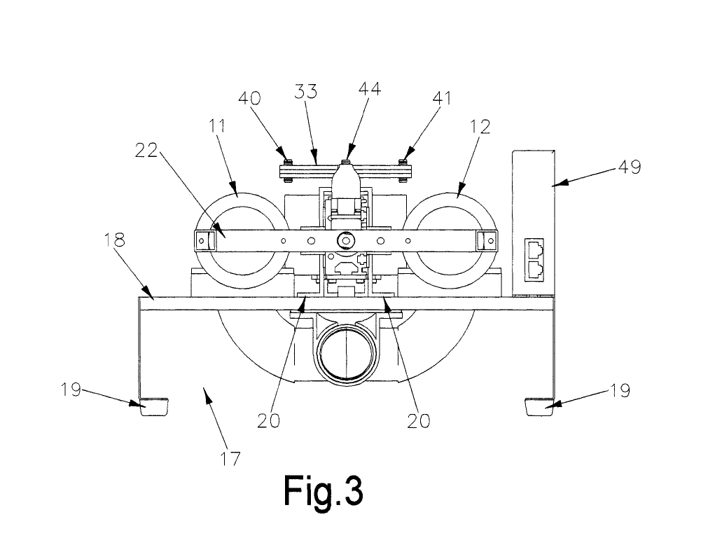

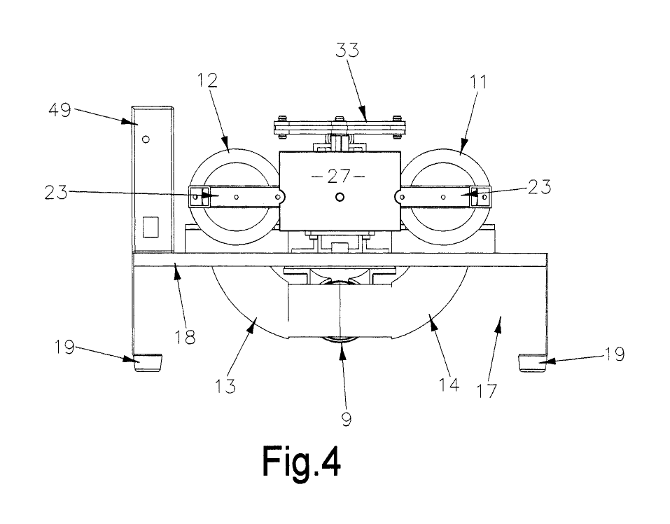

The bellows 11,12 and the linear motor 15 are mounted upon a base 17 which, as shown particularly in FIGS. 3 and 4, provides a flat supporting surface 18 which is rectangular in shape, and has a supporting foot 19 at each corner. The other side of the surface 18 supports the pairs of bellows 11,12, which are arranged as a first opposed pair of bellows 11, and a second opposed pair of bellows 12, spaced from the first pair of bellows and with the longitudinal axes of the bellows aligned and parallel to the longer edges of the surface 18. A pair of spaced rails 20 is mounted on the surface 18 between the pairs of bellows 11,12; the longitudinal axes of the rails 20 are parallel to the longitudinal axes of the aligned pairs of bellows 11,12.

The rails 20 carry the linear motor 15 and also carry the pressure bars 22,23, the outer ends which are arranged to bear against the closed ends of the corresponding bellows 11,12. The power supply to the motor 15 is of known type and is not shown in the drawings.

The motor 15 includes a stator 21 and a slider 25. Part of the stator is surrounded by cooling fins 26 to dissipate heat from the motor. The slider 25 is secured at one end to a counterweight 27 and the other end of the slider 25 (not visible) can slide freely into and out of the stator. The stator 21 and the slider 25 are coaxial. Movement of the slider 25 produces an equal movement of the pressure bar 23 and the counterweight 27.

The stator 21 is connected to the pressure bar 22 via the cooling fins 26 and a coupling 28 such that movement of the stator 21 produces an equal movement of the bar 22. The stator 21, cooling fins 26, coupling 28, and counterweight 27 can slide freely on the rails 20. The counterweight 27, slider 25 and pressure bar 23 together have the same mass as the stator 21, cooling fins 26, the coupling 28, and pressure bar 22, to balance the weight of the components as they move on the rails 20 and eliminate, or at least significantly reduce, any vibration. In other words, the mass of the stator 21 and everything which moves with it must balance the mass of the slider 25 and everything which moves with that.

A support arch 30, the inner arch of which is sufficiently large that the components of the linear motor 15 can pass through the arch freely, is rigidly secured to the supporting surface 18, and the top of the upper surface of the arch 30 carries the central pivot 31 of a lazy tongs linkage 33, which acts as a centering device: the linear motor 15 is, by nature of its design, balanced, and in operation the stator 21 and slider 25 would reciprocate towards or away from each other by an equal distance. However, it is possible for linear motors to “drift” off centre during use, or, if the ventilator is not on a completely horizontal surface gravity can move the motor off-centre: hence the need for a centering device.

The lazy tongs linkage 33 consists of two main arms 34,35 of equal length and arranged to form an X with the pivot 31 at the crossover point. On each side of the X two further arms 36,37,38,39 are connected together to form a V, with the ends of each V pivoted to the adjacent arms of the X by pivots 40,41,42 and 43, and a further pivot 44,45 at the apex of each V. The pivot 44 is secured to the upper surface of the cooling fins 26 and a pivot 45 is secured to the upper surface of the counterweight 27.

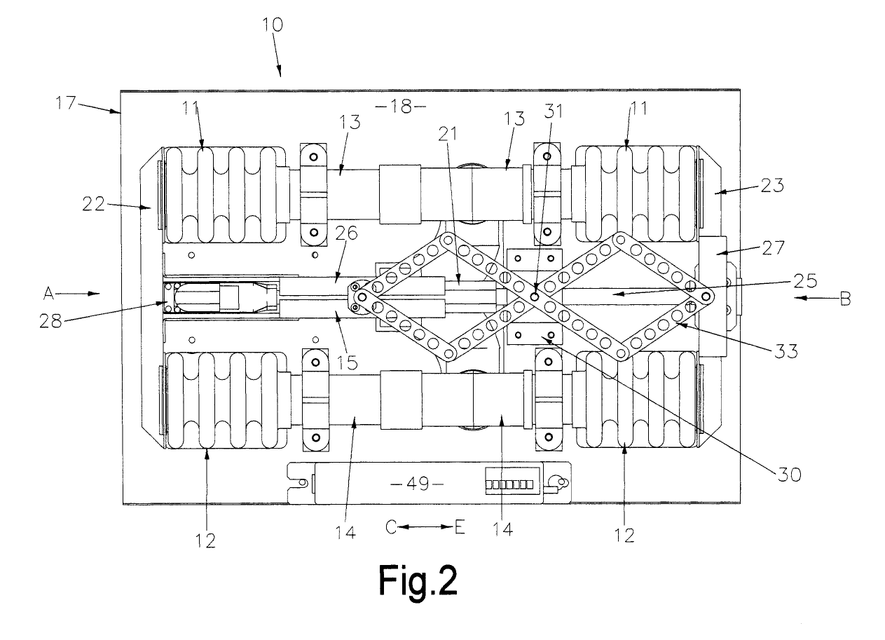

In use, when power is supplied to the linear motor 15, movement of the stator 21 (which of course also moves the cooling fins 26, coupling 28, and pressure bar 22) in either direction produces a mirror image movement of the slider 25, counterweight 27 and pressure bar 23. Thus, when the linear motor is activated and the stator 21 moves in either direction of arrow C or direction of arrow E (FIG. 2) the slider 25 and counterweight 27 move the same distance in the opposite direction. Movement of the stator 21 in the direction of arrow C expands the pairs of bellows 11,12 and movement of the stator 21 in the direction of arrow E compresses the pairs of bellows 11,12.

The bellows 11, 12 may be simple concertina type bellows made of any suitable material e.g. synthetic rubber or polyethylene. However differently shaped bellows could be used e.g. doughnut shaped bellows or D-shaped bellows.

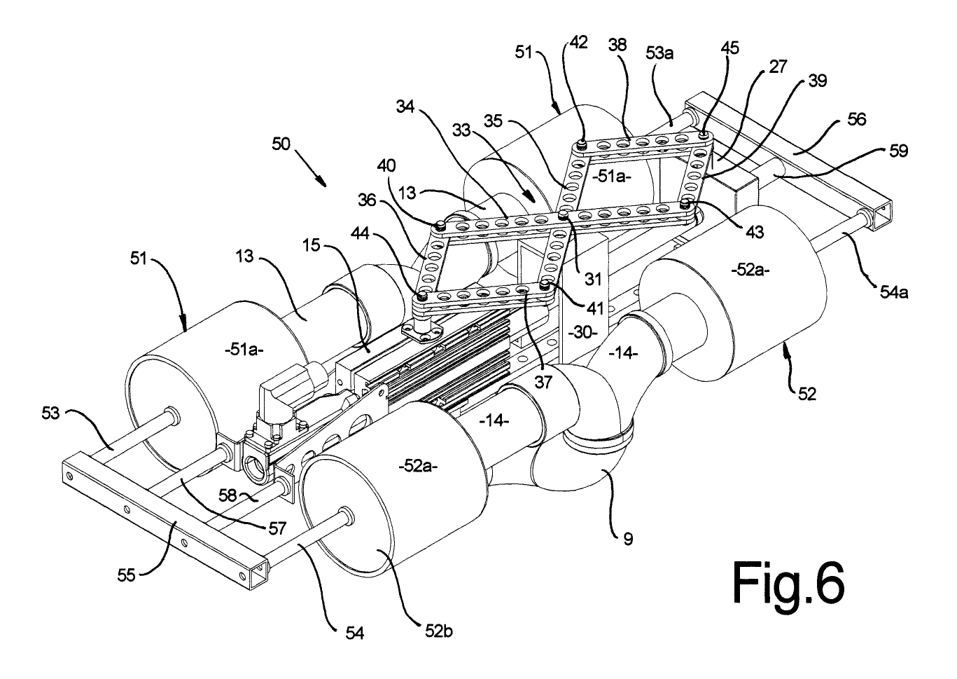

FIGS. 6 to 9 of the drawings show a second embodiment of the present invention. Many components of the second embodiment are identical to those of the first embodiment, and in these cases the same reference numeral is used. A ventilator 50 in accordance with a second embodiment of the present invention includes two pairs of opposed gas moving means in the form of pistons and cylinders 51, 52, each cylinder of each pair being connected at one end to an outlet tube 13, 14, which join into a common outlet 9.

The pairs of pistons and cylinders 51, 52 are spaced apart and a linear motor 15 is mounted between the pairs of pistons and cylinders. As with the first embodiment, the term “linear motor” means a linear induction motor. Any suitable linear motor in the known range of linear induction motors may be used; this may include a pair of linear motors, each acting on one opposing set of pistons and cylinders such that there is minimal net movement of the ventilator. As in the first embodiment, the linear motor 15 is powered by electricity, but the power connections are not shown in the drawings.

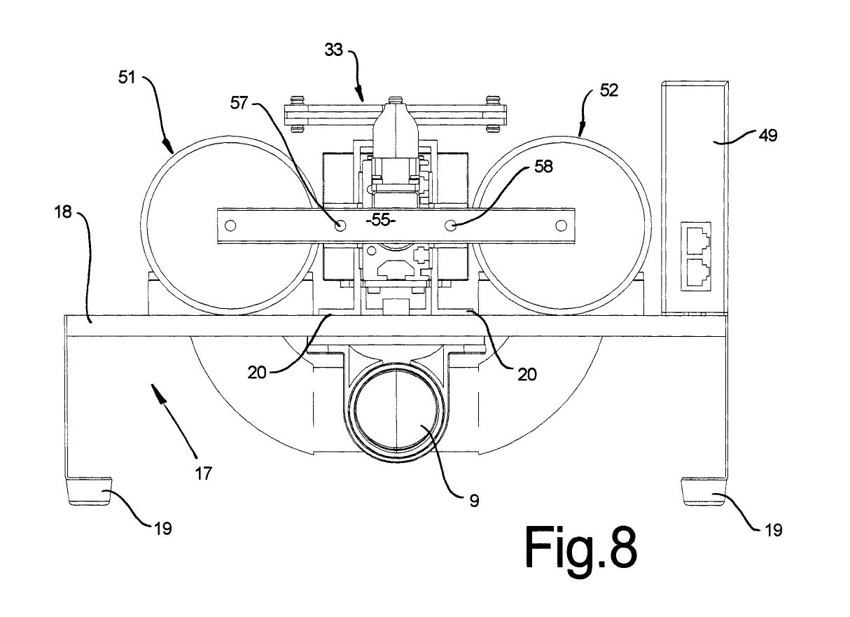

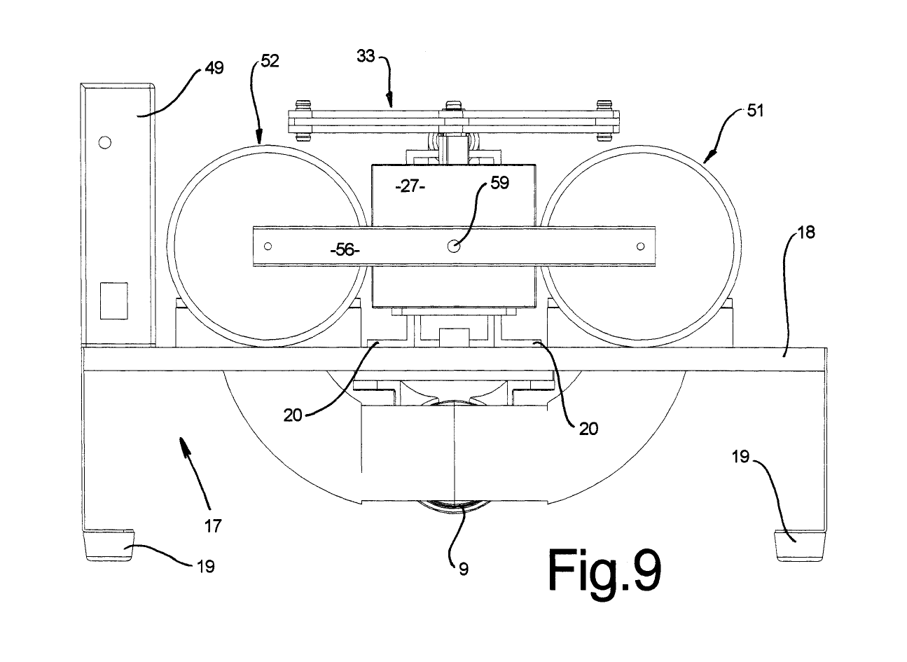

The pistons and cylinders 51, 52 and the linear motor 15 are mounted upon a base 17 which, as shown in FIGS. 8 and 9, provides a flat supporting surface 18 which is rectangular in plan and has a supporting foot 19 at each of the four corners. The side of the surface 18 opposite to the feet 19 supports the pairs of pistons and cylinders 51, 52 which are arranged as a first opposed pair of pistons and cylinders 51 and a second opposed pair of pistons and cylinders 52, with the two pairs of pistons and cylinders spaced apart and with the longitudinal axes of the pistons and cylinders aligned and parallel to the longer edges of the surface 18.

A pair of spaced rails 20 is mounted on the surface 18 between the pairs of pistons and cylinders 51, 52; the longitudinal axes of the rails 20 are parallel to the longitudinal axes of the aligned pairs of pistons and cylinders 51, 52. The rails 20 carry the linear motor 15.

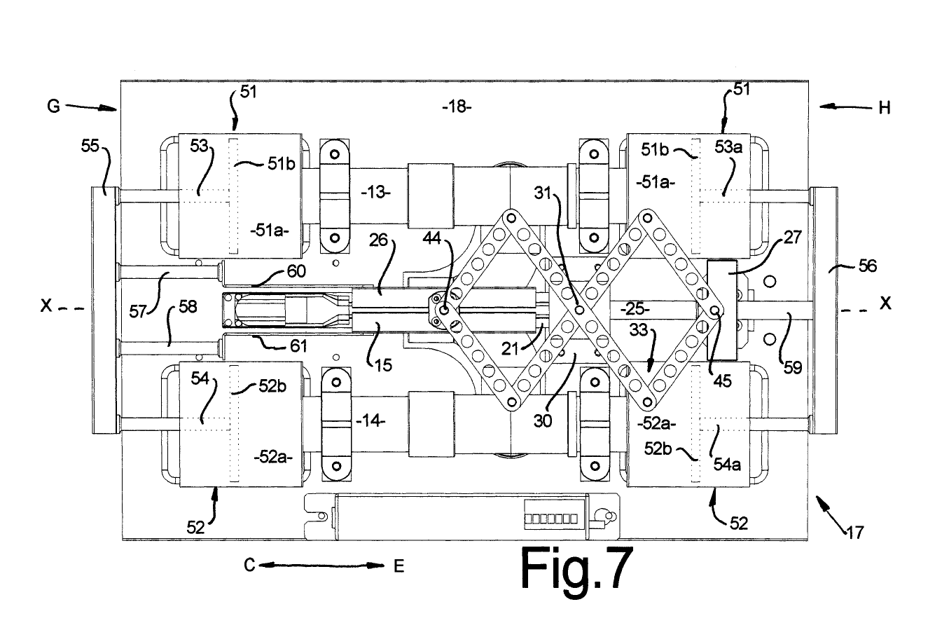

Each piston and cylinder 51, 52 consists of a cylinder 51a, 52a in which a piston 51b, 52b is arranged to reciprocate. FIG. 6 shows the pistons 51b, 52b at the end of their stroke (i.e. with the cylinders 51a, 52a at maximum capacity), whereas FIG. 7 shows the pistons 51b, 52b at about the mid-point of their stroke (i.e. with the cylinders 51a, 52a at about half capacity). Each piston is reciprocated within the cylinder by means of piston rods which form rigid connections between pushers 55, 56 and the respective pistons 51b, 52b of each pair of pistons and cylinders; pusher 55 is connected to piston rods 53 and 54; pusher 56 is connected to piston rods 53a and 54a. The cylinders 51a, 52a, of each pair are rigidly mounted on the supporting surface 18 and remain stationary while the pistons 51b, 52b, are reciprocated. This arrangement could be reversed: the cylinders 51a, 52a, could be arranged to reciprocate with the pushers while the pistons and piston rods remain stationary, but this is in general undesirable, because it requires the pushers 55, 56 to move a larger load, since the cylinders are considerably heavier than the pistons and piston rods combined, and also because the gas connection system for this arrangement is much more complicated.

The pistons, cylinders, and piston rods may be any of a wide variety of known constructions capable of providing a gas tight seal between piston and cylinder, to an acceptable level. The pistons may be provided with seals in known manner or a rolling diaphragm may be attached to the piston to minimise friction between the piston and cylinder and to ensure a gas tight seal. Alternatively, depending upon the type of gas being administered and the availability of that gas, it may be acceptable to tolerate a certain amount of leakage between piston and cylinder.

Each piston rod of the two opposed pairs of piston rods 53, 53a, 54, 54 a is rigidly secured at one end to the corresponding piston 51b, 52b and at the other end to the corresponding pusher 55, 56, so that force is transmitted directly from the pushers to the rods. Also rigidly connected to the pushers 55, 56 are connecting rods 57, 58 and 59. One end of each connecting rod 57, 58, 59 is rigidly connected to the adjacent pusher 55, 56 and the other ends of the connecting rods 57 and 58 are rigidly connected to a pair of spaced brackets 60, 61, which are rigidly secured to the sides of the cooling fins 26 of the motor 15. The other end of the connecting rod 59 is rigidly connected to a counterweight 27. The connecting rods 57, 58 are spaced apart, being equidistantly one on each side of the longitudinal axis X-X of the ventilator (FIG. 7); the connecting rod 59 is aligned with the longitudinal axis of the ventilator.

The motor 15 includes a stator 21 and a slider 25. Part of the stator is surrounded by cooling fins 26 to dissipate heat from the motor. The slider 25 is secured at one end to a counterweight 27 and the other end of the slider 25 (not visible) can slide freely into and out of the stator 21; the stator 21 and the slider 25 are coaxial. Movement of the slider 25 produces equal movements of the pusher 56, piston rods 53a, 54a secured to the pusher 56, the pistons 51b and 52b secured to the piston rods, connecting rod 59 and the counterweight 27.

The stator 21 is connected to the pusher 55 via the cooling fins 26, the brackets 60, 61, and the connecting rods 57, 58 such that movement of the stator 21 produces an equal movement of the pusher 55 and the associated piston rods 53, 54 with their associated pistons 51b and 52b. The stator 21, cooling fins 26, and counterweight 27 can slide freely on the rails 20. The counterweight 27, the pusher 56, the piston rods 53a, 54a and their associated pistons, and the connecting rod 59, together have the same mass as the stator 21, cooling fins 26, brackets 60, 61, pusher 55, connecting rods 57, 58, piston rods 53,54 and their associated pistons, to balance the weight of the components as they move on the rails 20 and eliminate, or at least significantly reduce, any vibration. In other words, the mass of the stator 21 and everything which moves with it is equal to the mass of the slider 25 and everything which moves with that.

A support arch 30, the inner arch of which is sufficiently large that the components of the linear motor 15 can pass through the arch freely, is rigidly secured to the supporting surface 18, and top of the upper surface of the arch 30 carries the central pivot 31 of a lazy tongs linkage 33, which acts as a centering device for the reasons discussed with reference to the first embodiment.

The lazy tongs linkage 33 consists of two main arms 34, 35 of equal length and arranged to form an X with the pivot 31 at the crossover point. On each side of the X two further arms 36, 37, 38, 39 are connected together to form a V, with the ends of each V pivoted to the adjacent arms of the X by pivots 40, 41, 42 and 43, with a further pivot 44, 45 at the apex of each V. The pivot 44 is secured to the upper surface of the cooling fins 26 and the pivot 45 is secured to the upper surface of the counterweight 27.

In use, movement of the stator 21 (which of course also moves the cooling fins 26, brackets 60, 61, connecting rods 57, 58, piston rods 53, 54 and their pistons, and pusher 55) in either direction produces a mirror image movement, (i.e. the same distance in the opposite direction) of the slider 25, counterweight 27, connecting rod 59, piston rods 53a, 54a, and their pistons, and pusher 56. Thus, when the linear motor is activated and the stator 21 moves in either direction of arrow C or direction of arrow E (FIG. 7) the slider 25 and counterweight 27 move in the opposite direction by the same distance. Movement of the stator 21 in the direction of arrow C provides the induction stroke for the pairs of cylinders and pistons 51, 52 and movement of the stator 21 in the direction of arrow E provides the compression stroke for the pairs of cylinders and pistons 51, 52. In each induction stroke, the piston moves so as to increase the volume enclosed between the wall of the cylinder and the piston, and thus draws gas into the cylinder; in the compression stroke the piston moves so as to decrease the volume enclosed between the wall of the cylinder and the piston, and thus expels gas from the cylinder.

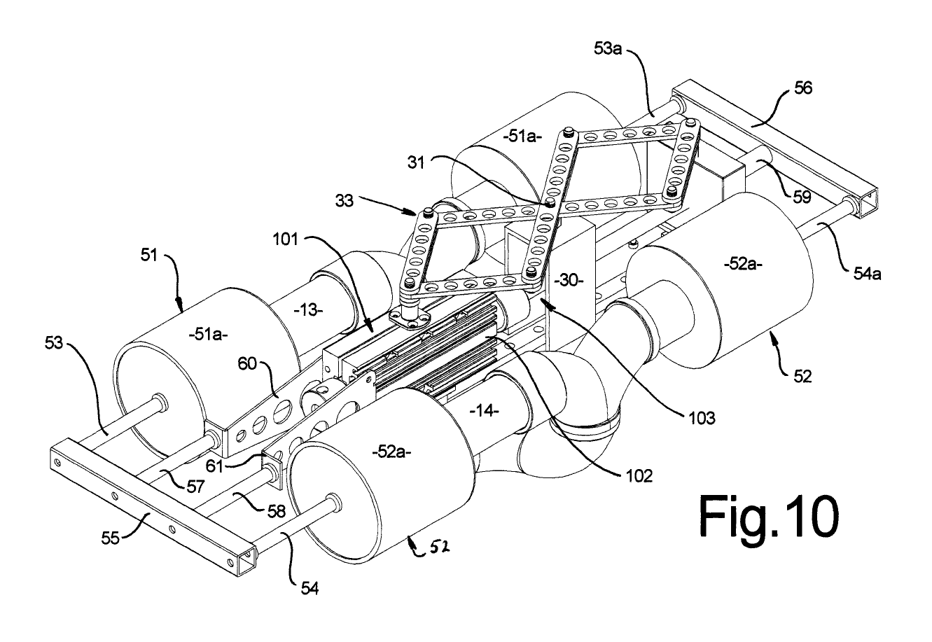

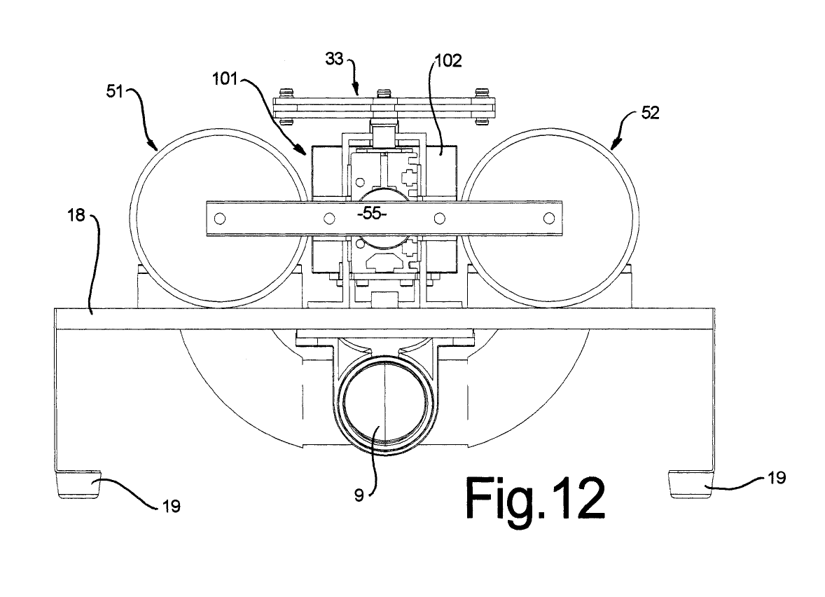

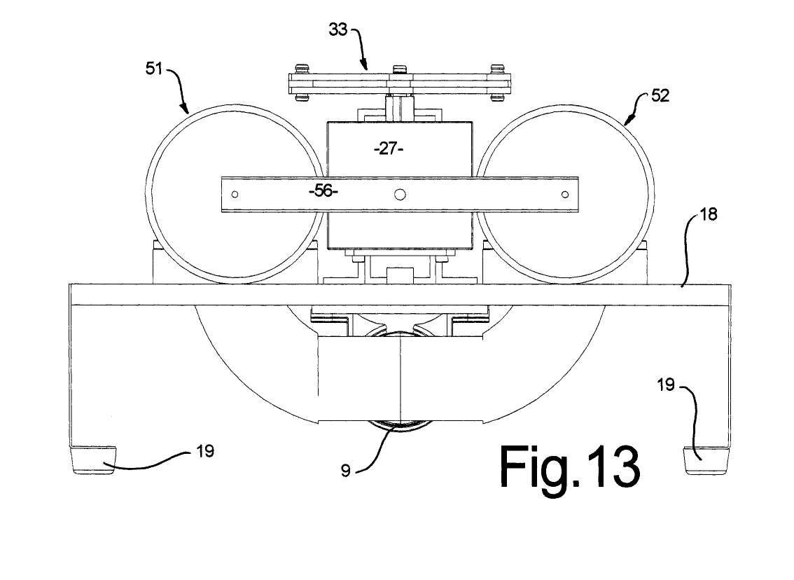

FIGS. 10 to 13 show a third embodiment of the present invention, in which the linear motor 15 is replaced by a pneumatic or hydraulic ram 101. The embodiment shown in FIGS. 10 to 13 is identical to that described with reference to FIGS. 6 to 9 except for the substitution of the linear motor 15 by a pneumatic or hydraulic ram 101, and therefore only this feature will be described in detail; the same reference numerals as in FIGS. 6 to 9 are used where the components are the same.

The pneumatic or hydraulic ram 101 may be of any suitable known type and consists, in known manner, of a cylinder containing a piston mounted on a piston rod; the cylinder is fitted with pneumatic/hydraulic inlets and outlets in known manner; these are not shown in the drawings for reasons of clarity. The ram 101 is mounted on the rails 20 and is surrounded by cooling fins 102 to which brackets 60, 61 are attached to form a load transmitting connection between the ram 101, the cooling fins 102, the connecting rods 57, 58, the piston rods 53, 54 and their pistons, and the pusher 55. The pneumatic or hydraulic cylinder of the ram 101 has a piston rod 103 one end which is rigidly secured to the counterbalance 27 and the other end of which (not visible) carries a piston mounted within the cylinder in known manner.

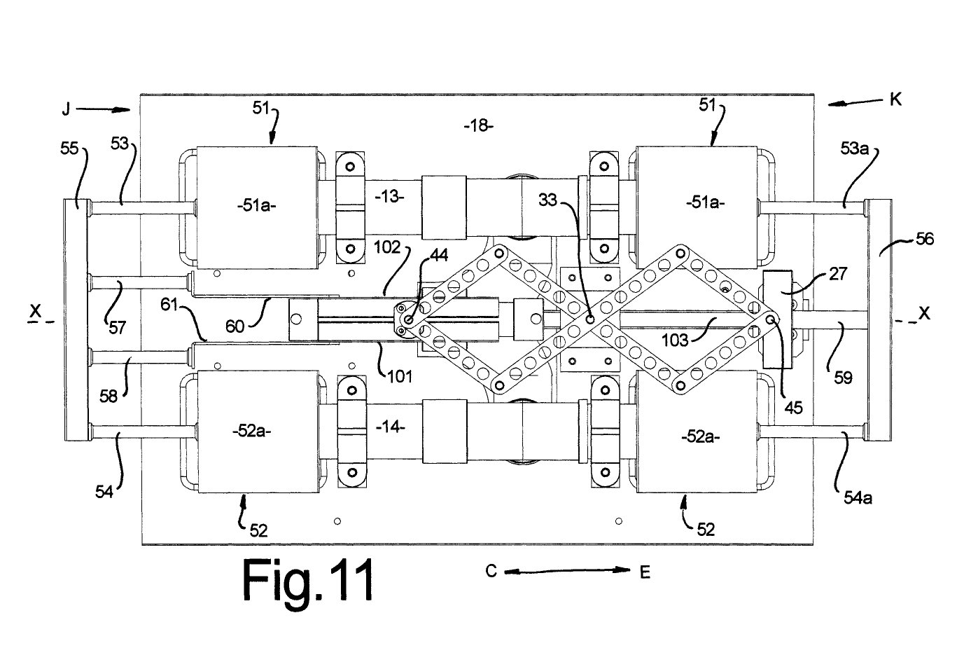

When fluid is admitted to the ram 101, the piston rod 103 is moved in the direction of arrow E in FIG. 11, and thus moves the counterweight 27, connecting rod 59, pusher 56, and associated piston rods 53a, 54a, and their pistons in the same direction. There is an equivalent movement, in the direction of arrow C, of the ram 101, cooling fins 102, brackets 60, 61, connecting rods 57, 58, pusher 55 and associated piston rods 53, 54 and their pistons. This provides an induction stroke for the pairs of cylinders and pistons 51, 52. When fluid is withdrawn from the ram 101, the piston rod 103, counterweight 27, connecting rod 59, pusher 56 and associated piston rods 53a, 54a and their pistons move in the direction of arrow C, and the ram 101, cooling fins 102, brackets 60, 61, connecting rods 57, 58 pusher 55 and associated piston rods 53, 54 and their pistons move in the direction of arrow E, providing a compression stroke for the pairs of cylinders and pistons 51, 52. As with the embodiment described with reference to FIGS. 6 to 9, the components which are moved in opposite directions have the same mass so as to eliminate, or at least significantly reduce, any vibration. As with the embodiments one and two, the lazy tongs linkage 33 acts as a centering device.

In the above described examples, movement means in the form of bellows had been described with reference to a ventilator in which the balanced drive is a linear motor, but it will be appreciated that the balanced drive could also be a pneumatic or hydraulic ram i.e. with bellows substituted for the cylinders and pistons described with reference to FIGS. 10 to 13.

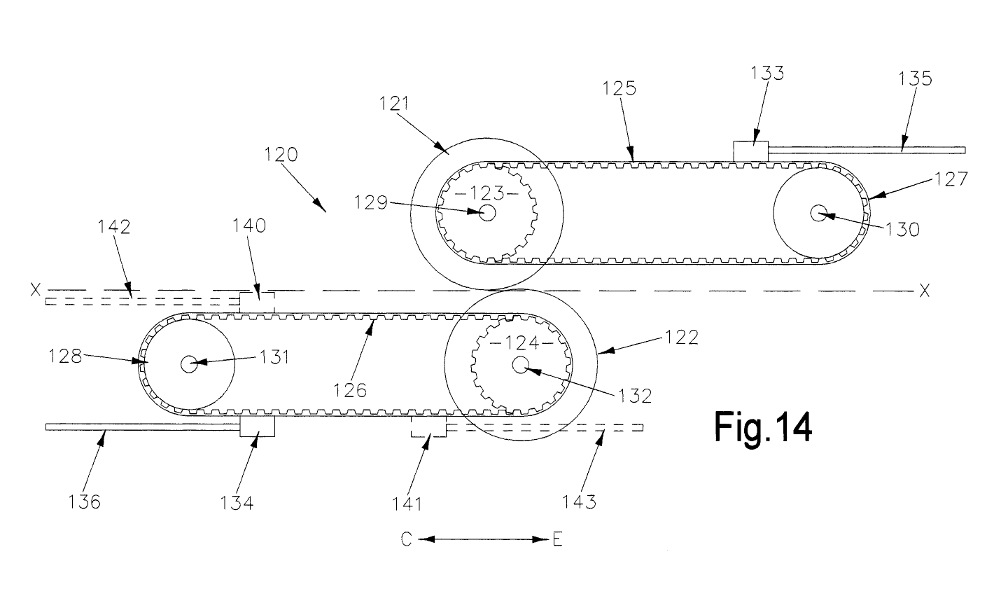

Another type of balanced drive which can be used instead of a linear balance drive, is shown in FIG. 14, which depicts two possible configurations of a rotary balanced drive 120.

FIG. 14 shows two identical stepper motors 121, 122, each having a driven sprocket 123, 124, the outer circumference of which is toothed, and is in driving engagement with a toothed drive belt 125, 126.

Each drive belt 125, 126 extends around a pulley 127, 128. The stepper motors 121, 122 are mounted one on each side of the longitudinal axis X-X of the ventilator, and the centres of rotation 129, 130/131, 132 of the sprockets and pulleys associated with each stepper motor lie on lines parallel to said longitudinal axis.

The pulleys 127, 128 may be smooth-surfaced, as shown, or may be toothed for a more positive drive.

A carrier 133, 134 supporting a connecting rod 135, 136 is mounted on each drive belt 125, 126, with the connecting rod extending away from the corresponding sprocket.

The free end of each connecting rod 135, 136 is rigidly connected to the pressure bars 22, 23 respectively (in the embodiment of FIGS. 1-4) or the pushers 55, 56 respectively (in the embodiment of FIGS. 6-10), so that movement of the drive belt 125 in the direction of arrow E and corresponding movement of the drive belt 126 in direction of arrow C, expands the bellows 11, 12, or, in the case of pistons and cylinders 51, 52, increases the volume of the cylinders; the opposite movement of the drive belts compresses the bellows/decreases the volume of the cylinders.

The drives of the stepper motors 121, 122 are synchronised, to produce a uniform effect on all of the gas moving means.

An alternative to the above-described drive is a single balanced rotary drive, indicated in broken lines in the lower part of FIG. 14. In this variant, a single drive belt 126 carries two opposed carriers 140, 141 and corresponding connecting rods 142, 143. The free ends of the connecting rods 142, 143 are connected to the pressure bars 22, 23 or the pushes 55, 56, and operate in the manner described above.

In a further variant, toothed rods could engage the sprockets 123, 124 directly, and replace the drive belt, the carrier and the connecting rod, being connected directly to the pressure bars or pushers.

It will be appreciated that if a single stepper motor is used, it may be necessary to weight the pulley 124 to compensate for the greater weight of the stepper motor.

The stepper motor(s) and associated pulleys do not require the rails 20, and are mounted on the surface 18. The stepper motor(s) are electrically powered, in known manner.

The operation of the ventilator of the present invention is described with particular reference to the embodiment of FIGS. 1 to 4, but the second and third embodiments operate in the same way, except that the action of the pairs of bellows 11, 12 is replaced by the action of the pairs of pistons and cylinders 51, 52.

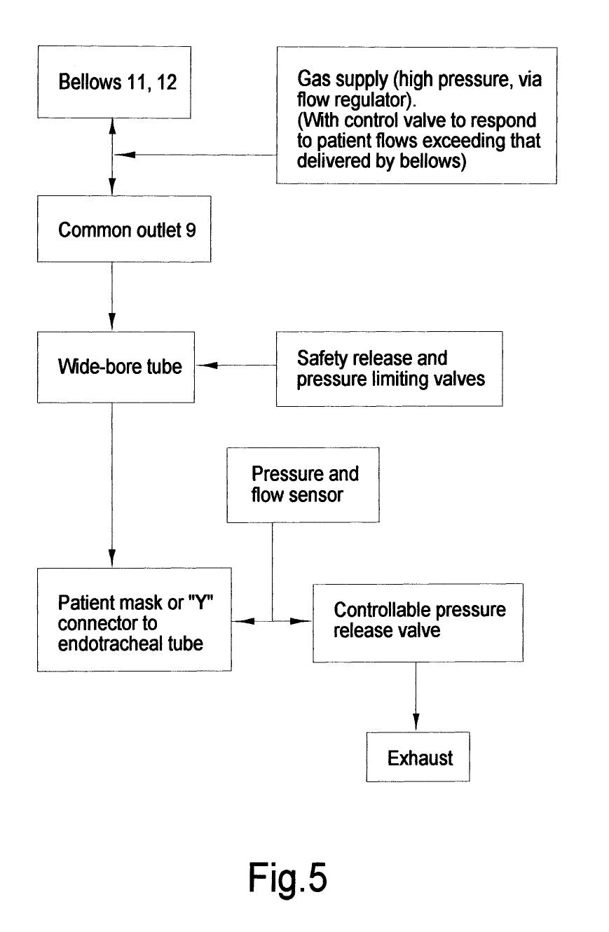

As shown diagrammatically in FIG. 5, the pairs of bellows 11,12 (or pistons and cylinders 51, 52) are connected to the common outlet 9, then to a high efficiency particulate air (HEPA) filter, and then to a (preferably) wide-bore hose, which conducts the oscillatory pressure and variable, controlled gas flows and gas pressures to the patient according to the mode of ventilation. The HEPA filter prevents contamination of the bellows/cylinders and internal apparatus of the ventilator, and also protects the patient from any particulate debris which may occur during the operation of the ventilator. The wide bore hose terminates at either a mask worn by the patient or at a Y connector attached to an endotracheal tube, where the distal end lies within the patient's trachea.

The common outlet 9 and any tubing in the patient delivery system is preferably wide bore tubing. Typically, the wide bore tubing has a diameter of 35-45 mm, to allow a rapid response to changes in flow and pressure sensed close to the patient's airway, which permits better synchrony and may reduce the imposed work of breathing. The wide bore tube will allow propagation of the high frequency oscillatory pressure changes to be conducted without significant loss of energy. The wide bore tube is preferably transparent, light, and flexible, but with walls providing a high level of stiffness (high elastances). Preferably the tubing is reinforced with wire.

The type of patient delivery system used depends upon the type of ventilation being provided—for example if CPAP is being provided to a patient who can breathe, the system may include a mask incorporating an exhaust valve, whereas if ventilation is being provided to a patient who cannot breathe, an endotracheal tube may be required. A sensor is located in the patient delivery system to monitor gas flow and/or measure exhaust flow from the patient.

A supply of gas (which may be air, or oxygen, or oxygen enriched air, or any other specified gas mixture) also is connected to the common outlet 9 such that when the bellows are expanded, the gas is drawn into the bellows, and is then expelled from the bellows, under pressure, when the bellows are compressed. In the second and third embodiments, gas is drawn into the cylinders during the induction stroke, and expelled from the cylinders during the compression stroke. The gas supply is pressurised, and is supplied via a flow regulator. The gas flow preferably is controlled using a solenoid valve, which delivers pressurised gases at a controlled rate to the common outlet 9. The airway pressure (i.e. the pressure in the gas delivery circuit next to the mask or the Y connector) is controlled by an expiratory control valve through which the patient's expired gases are exhausted. The resistance of the expiratory control valve is varied to maintain constant or variable pressures and volumes delivered to the patient.

Assisted spontaneous breathing (partially controlled) and controlled mandatory ventilation (fully controlled) are effected by controlling both the expiratory control valve and movement of the pistons to generate desired flows and pressures, which may be synchronised with the patient's own breathing efforts.

If the patient inspires faster than the flow being delivered by the ventilator, the pressure in the airway will fall. This will initially be met by actuation of the bellows/pistons and cylinders to compress a greater volume. However, if the demand exceeds the volume capacity of the bellows/cylinders, (approximately 1500 mL) the solenoid valve controlling the pressurised fresh gas flow will open further to ensure the desired delivery pressures and volumes are met.

The pressure of the gas supplied to the patient delivery system depends upon the pressure applied to the bellows/pistons by the pressure bars 22,23, or pushers 55, 56 respectively and the frequency with which the bellows are compressed/expanded or the pistons are moved between compression and induction strokes, (and hence the frequency of the “breaths” supplied to the patient) depends upon the rate at which the balanced drive (i.e. linear motor 15 or pneumatic or hydraulic ram 101) is set to reciprocate. The rate of reciprocation of the balanced drive is regulated using a control box 49 mounted along one side of the surface 18 (not shown in FIGS. 1, 6, 10-13).

The expired gases from the patient are exhausted through a one-way pressure release valve, which is controlled either pneumatically or electromagnetically. In the simplest embodiment the valve may be controlled using fixed or variable spring or by a pneumatic system in either the CPAP or HFV modes. A controllable pressure-release valve is required for all other forms of assisted or controlled ventilation. Pressure and flow sensors are provided at or near the mask or the Y connector. The wide-bore tube also is provided with safety release and pressure-limiting valves.

The balanced drive is controlled by software algorithms written for each specific ventilation mode. The control input to the algorithms may be either directly entered by the user or received from sensors monitoring the apparatus and breathing circuit, such as flow and pressure sensors.

The algorithms control both the degree of movement of the balanced drive (and thus the distance through which the bellows or pistons are moved) and the drive's rate of reciprocation (and thus the frequency with which the bellows or pistons are moved). Different types of ventilation can be provided by the ventilator simply by adjusting the degree of movement and the rate of reciprocation of the balanced drive: to provide CPAP or ventilation for a spontaneously breathing patient, the reciprocation of the balanced drive must be synchronised with the patient's own breathing, for example using a pressure/flow sensor in the patient delivery system to provide the control parameters for the linear motor. For a patient who is not spontaneously breathing, the patient's breathing rate is set by the ventilator, with the pressure levels and volume delivered determined by the patient's lung size and condition.

The balanced drive can readily be set to deliver high-frequency ventilation, since the drive can reciprocate at high speed if required. Further, if it is considered advisable to superimpose a high-frequency ventilation on a standard ventilation pattern, this can be achieved by superimposing a high-speed short stroke reciprocation of the drive on the normal reciprocation of the drive, or by using the ventilator of the present invention in combination with a standard ventilator, using the ventilator of present invention to provide the high-speed short stroke reciprocation required for high-frequency ventilation, in combination with standard mode ventilation.

1. A ventilator which includes: at least one pair of reciprocating opposed gas moving means, the internal volume of which is arranged to be reduced and enlarged by a balanced drive, said gas moving means being arranged to move gas at a predetermined pressure and/or volume to a delivery tube; control means for regulating the speed and the distance of movement of the drive; wherein the masses of all moving parts are balanced so as to minimise the vibration of the ventilator in use.

2. The ventilator as claimed in claim 1, wherein said gas moving means is selected from the group consisting of: bellows; cylinders and pistons.

3. The ventilator as claimed in claim 1 or claim 2, wherein said balanced drive is selected from the group consisting of: a linear balanced drive; a rotary balanced drive.

4. The ventilator as claimed in claim 1, wherein said balanced drive is a linear balanced drive which is arranged to reciprocate to reduce and enlarge the internal volume of said gas moving means and which is selected from the group consisting of: a linear induction motor; a pneumatic ram; a hydraulic ram.

5. The ventilator as claimed in claim 4, wherein said balanced drive is arranged in two parts: a first part which is connected by first connection means to one gas moving means of the or each pair of gas moving means, and a second part which is connected by second connection means to the other gas moving means of the or each pair of gas moving means; said the first and second parts being such that linear movement of either part of said balanced drive producers an equal but opposite movement of the other part of said balanced drive.

6. The ventilator as claimed in claim 5 wherein said first and second parts are linked by a centering device to maintain correct centering in use.

7. The ventilator as claimed in claim 6 wherein said centering device is a lazy tongs linkage.

8. The ventilator as claimed in claim 5, wherein said balanced drive is mounted upon rails and is arranged to move upon said rails when reciprocating.

9. The ventilator as claimed in claim 5 or claim 8, wherein said at least one pair of opposed gas moving means comprises two pairs of opposed gas moving means, arranged with the longitudinal axis of the gas moving means of each pair aligned with each other, said pairs being spaced apart and having said balanced drive mounted between said pairs.

10. The ventilator as claimed in claim 5, wherein the mass of said first part of said balanced drive and said first connection means is substantially equal to the mass of said second part of said balanced drive and said second connection means, so as to minimise vibration of the ventilator in operation.

11. The ventilator as claimed in claim 1, wherein said gas moving means comprise cylinders and pistons, and wherein said at least one pair of opposed gas moving means comprises two pairs of opposed pistons and cylinders arranged with the longitudinal axes of the cylinders of each pair aligned with each other, said pairs being spaced apart and having a balanced drive in the form of a linear induction motor mounted equidistantly between said pairs; said linear induction motor including a stator and a slider coaxial with the stator; wherein said stator and said slider are arranged such to be reciprocated relative to each other and such that linear movement of said stator or said slider produces an equal but opposite movement of said slider or said stator; said stator being connected to one piston of each pair of pistons by first connecting means arranged such that reciprocation of said stator reciprocates each said piston within the corresponding cylinder; and said slider being connected to the other piston of each pair of pistons by second connecting means arranged such that reciprocation of said slider reciprocates each said piston within the corresponding cylinder.

12. The ventilator as claimed in claim 11, wherein said second connecting means incorporates a counterweight.

13. The ventilation delivery system which includes a ventilator as claimed in claim 1 or claim 11, a gas supply, a gas delivery hose, and patient gas delivery means.

14. The system as claimed in claim 13, wherein said gas delivery hose is a wide bore hose.

15. The system as claimed in claim 13 wherein the patient delivery means is selected from the group consisting of: a mask; an endotracheal tube connector.
